(19) United States
(12) Patent Application Publication
(10) Pub. No.: US 2008/0140022 A1
(43) Pub. Date: Jun. 12, 2008

(54) Coated Cannula with Protective Tip for Insertion Into a Patient

(75) Inventors: John Durward Pond, Germantown, TN (US); Robert B. Rice, Southaven, MS (US)

Correspondence Address: COATS & BENNETT/MEDTRONICS, 1400 CRESCENT GREEN, SUITE 300, CARY, NC, 27518, US

(73) Assignee: WARSAW ORTHOPEDIC, INC., Warsaw, IN (US)

(21) Appl. No.: 11/608,585

(22) Filed: Dec. 8, 2006

(51) Int. Cl. A61M 5/32

(52) U.S. Cl. 604/272

(57) ABSTRACT

The present application is directed to a cannula for insertion into a patient. The cannula includes a first distal section and a second proximal section with a hollow interior extending through each. The first section is positioned distally of the second section and includes a larger outer diameter. A coating is applied to the second section. An outer diameter of the coating is smaller than or equal to the outer diameter of the first section. This design prevents the coating from detaching from the second section during insertion of the cannula into the patient. In one embodiment, the cannula acts as a guide for inserting the coating into the patient and the first section protects the leading edge of the coating.

### BACKGROUND

The present invention is directed to a cannula for insertion into a patient and, more particularly, to a coated cannula with a protective tip to maintain attachment of the coating.

Various medical procedures require a physician to examine a patient's tissue or bone, obtain a sample of a patient's tissue or bone, or penetrate to the bone marrow cavity to extract bone, bone marrow or bone marrow cavity fluids. The procedures require the physician to use a sharpened instrument to penetrate the tissue, or the hard, outer layer of the bone. The procedures require the instrument to have a combination of attributes including rigidity to prevent bending and breaking while being inserted into the bone, and be of a minimum size to prevent unnecessary damage to the bone and surrounding tissue.

The sharpened instrument may include a coating attached to an outer surface. The coating may be necessary for various reasons, including lubricity to facilitate insertion into the patient, and electrical insulation when the instrument is used in combination with electrical procedures. A problem with prior art devices is the coating detaches from the instrument during insertion into the bone. This occurs because the density of the bone overcomes the adhesion of the coating to the instrument causing the coating to tear or peel away. The failure of the coating is more likely when the procedure requires numerous insertions into the tissue and/or bone.

Neuro integrity monitoring is one example of a procedure that uses a sharpened instrument for bony insertion. Neuro integrity monitoring is an intraoperative procedure that penetrates a probe into a patient's bone. An electrical current is transmitted through the probe to determine surgical data such as proximity to nerves, motor nerve irritation and positioning-related neuropathy, and spinal cord motor conduction integrity. Probe is inserted into the bone through a cannula that is previously inserted into the bone. The cannula includes a dielectric coating that forms insulation barrier between the probe and the surrounding tissue. Maintaining the dielectric coating attached to the cannula is important for obtaining accurate results.

### SUMMARY

The present application is directed to a cannula for insertion into a patient. The cannula may include a first section and a second section with a hollow interior extending through each. The first section is positioned distally of the second section and may include a larger outer diameter. A coating may be applied to the second section. An outer diameter of the coating may be smaller than or equal to the outer diameter of the first section. This design may provide for the first section to prevent the coating from detaching from the second section during insertion of the cannula into the patient. In one embodiment, the cannula acts as a guide for inserting the coating into the patient and the first section protects the leading edge of the coating.

### BRIEF DESCRIPTION OF THE DRAWINGS

FIG. 1 is a side view of a cannula according to one embodiment.

FIG. 2 is a partial cross-sectional view taken along line II-II of FIG. 1.

FIG. 3 is a cross-sectional view of a cannula according to one embodiment.

FIG. 4 is a cross-sectional view of a cannula according to one embodiment.

FIG. 5 is a cross-sectional view of a cannula according to one embodiment.

FIG. 6 is a cross-sectional view of a cannula according to one embodiment.

### DETAILED DESCRIPTION

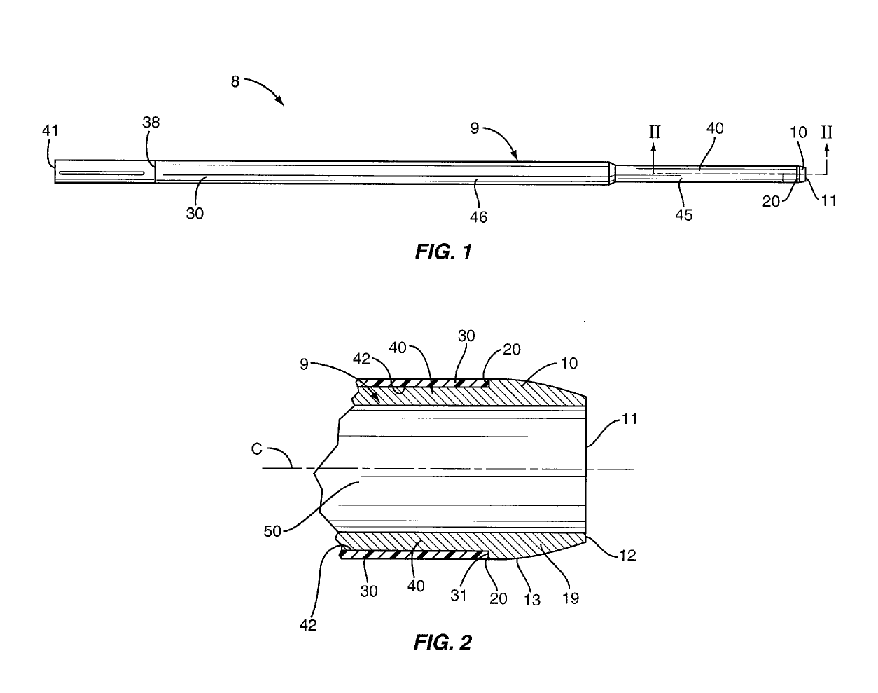

The present application is directed to a coated cannula for insertion into a patient. FIGS. 1 and 2 illustrate one embodiment of a cannula 8 that includes a body 9 with a shoulder 20 formed between first section 10 and a proximal second section 40. A coating 30 is applied to the second section 40. An outer diameter of the first section 10 is greater than or equal to an outer diameter of the coating 30. Therefore, the first section 10 acts as a guard during insertion of the cannula 8 into the patient to shield the coating 30 and prevent removal from the second section 40.

The body 9 is constructed of a rigid material to prevent bending or breaking during insertion into the patient. As illustrated in FIGS. 1 and 2, body 9 extends between a distal end 11 and a proximal end 41. Body 9 includes a first section 10, shoulder 20, and a second section 40. Body 9 includes a hollow interior 50 that extends along the length from the distal end 11 to the proximal end 41. A width of the hollow interior 50 may be constant or may vary along the length of the body 9. Body 9 may be constructed from various materials including but not limited to stainless steel, titanium, and aluminum. Body 9 may be constructed as a single element, or may be constructed from two or more separate elements that are attached together.

In one embodiment, each of the first and second sections 10, 40 includes a substantially circular cross-sectional shape. The sections 10, 40 may also include other cross-sectional shapes such as oval and polygonal. Further, the first section 10 may include different cross-sectional shape than the second section 40.

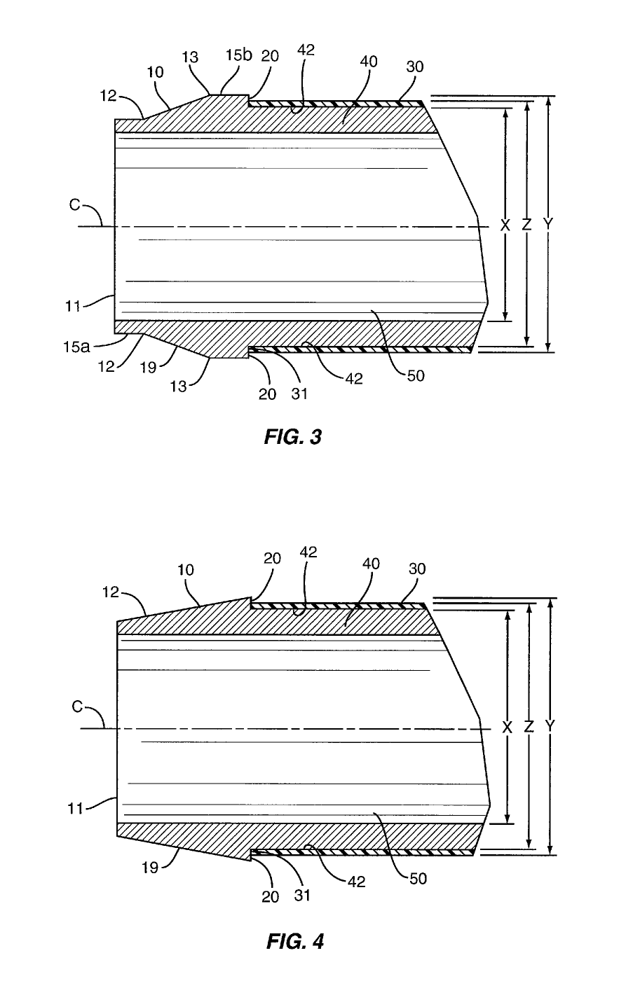

The first section 10 may include a tapered section 19 that facilitates insertion into the patient. Tapered section 19 extends between a first longitudinal position 12 with a reduced outer diameter and a second longitudinal position 13 with an expanded outer diameter. The first longitudinal position 12 may coincide with the distal end 11 of the body 9 as illustrated in FIGS. 1 and 2, or may be spaced inward in a proximal direction from the distal end 11 as illustrated in FIG. 3.

In one embodiment, the entire length of the first section 10 is tapered as illustrated in FIG. 4. The taper extends from the first longitudinal position 12 at the distal end 11 to the second longitudinal position 13 at the shoulder 20. First section 10 may also include one or more non-tapered sections 15. FIG. 3 illustrates an embodiment with a first non-tapered section 15a between the distal end 11 and the first longitudinal position 12. A second non-tapered section 15b is positioned between the second longitudinal position 13 and the shoulder 20. FIG. 6 illustrates a non-tapered section 15 between two tapered sections. The length of the non-tapered sections 15 may vary depending upon the specific embodiment.

The second section 40 extends proximally from the first section 10. As illustrated in FIGS. 3 and 4, an outer diameter X of the second section 40 is less than an outer diameter Y of a proximal section of the first section 10. The differences in the outer diameters X, Y may be relatively large as illustrated in FIGS. 3 and 4, or relatively small as illustrated in FIG. 2. The term “diameter” is used herein to mean the size of the element by a straight line passing through a center of the cross-sectional shape. The term “diameter” is used to include circles, as well as other cross-sectional shapes.

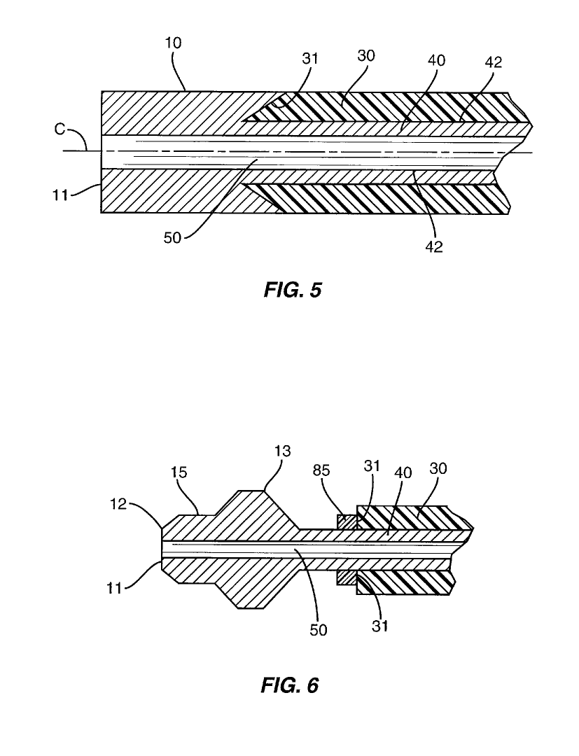

The shoulder 20 is formed at the junction between the first and second section 10, 40. Shoulder 20 extends between an outer surface of the first section 10 and an outer surface of the second section 40. Shoulder 20 may be aligned at various angular positions relative to a longitudinal centerline C of the body 9. In one embodiment illustrated in FIGS. 2, 3, and 4, shoulder 20 is aligned substantially perpendicular to the centerline C. FIG. 5 illustrates an embodiment with shoulder 20 aligned at a non-perpendicular angle.

Coating 30 is adhered to the outer surface 42 of the second section 40. Coating 30 may be required on the cannula 8 for various reasons. In one embodiment, coating 30 is a dielectric insulator that acts as a barrier to prevent shunting between a monitoring instrument placed within the interior 50 and the surrounding tissue and/or bone. The coating 30 may be used for other functions in electrical surgical applications including cutting, cauterizing, and stimulization. Coating 30 may further provide lubrication or friction resistance to facilitate insertion and removal of the cannula 8. Coating 30 may also provide numerous other functions including but not limited to corrosion resistance, heat resistance, protection against patient sensitivity, friction resistance, antimicrobial protection, anti-migration, abrasion resistance, anti-reflection, flexation reduction, as a means to protect the body 9 and extend the life of the cannula 8, and as a color-code identification.

The thickness of the coating 30 is limited such that an outer diameter Z of the coating 30 is less than or equal to the outer diameter Y of the first section 10 as illustrated in FIGS. 3 and 4. This sizing protects the coating 30 during insertion of the cannula 8 into the patient. The first section 10 acts as a shield to form an opening in the tissue and/or bone for insertion of the proximal sections of the cannula 8.

In one specific embodiment, the body 9 acts as a guide for inserting the coating 30 into the patient. The second section 40 is a holder for the coating 30. The first section 10 protects the leading edge 31 of the coating 30 from separating from the second section 40.

A variety of different coatings 30 may be applied to the second section 40. Examples of coatings 30 include but are not limited to TEFLON, nylon including RILSAN, PEEK, PTFE, plastics, xylan, HALAR, TEFZEL, fluoropolymer, managed surface finishes, phenolics, epoxies, vinyls, and acrylics. Coating 30 may also be an anodized or galvanized layer formed on the exterior of the second section 40. The coating 30 may also be applied to the second section 40 in a number of different manners including but not limited to liquid dispersion, powder coating, dip coating, shrink wrap, molding, hard facing, metalizing, electric arc, thermal spray, plasma spray, high-velocity oxygen fuel, and adhesive.

In one embodiment, the leading edge 31 (i.e., the distal edge) of the coating 30 contacts the shoulder 20 as illustrated in FIGS. 2, 3, 4, and 5. In another embodiment as illustrated in FIG. 6, the leading edge 31 is spaced away from the shoulder 20. In one embodiment, a spacer 85 is positioned adjacent to the shoulder 20 to space apart the coating 30.

Coating 30 may be applied to a limited length or the entirety of the second section 40. FIG. 1 illustrates an embodiment with the coating 30 being applied to the second section 40 from the shoulder 20 to a longitudinal position 38. The remaining length of the second section between position 38 and proximal end 41 is not coated.

Second section 40 may include various shapes and sizes. In one embodiment, second section 40 includes a substantially constant outer diameter. In another embodiment as illustrated in FIG. 1, second section 40 includes a first proximal length 45 adjacent to the first section 20 with a reduced outer diameter X as explained above. A second proximal length 46 includes a larger outer diameter.

The hollow interior 50 is sized to receive various instruments. In one embodiment, interior 50 is sized to receive a stylet that includes a pointed tip or cutting edge for penetrating into the bone or tissue of the patient. The stylet may also function as an electrical probe as part of a neuro integrity monitoring system. Interior 50 may include substantially the same width along the length of the body 9, or may include a varying width.

One context for using the cannula 8 is during spinal treatments. The cannula is sized for insertion into a vertebral member or spinal canal along the various regions of the spine, including the cervical, thoracic, lumbar and/or sacral regions. It should be further understood that cannula 8 also may be used in other non-spinal contexts.

The term “distal” is generally defined as in the direction of the patient, or away from a user of a device. Conversely, “proximal” generally means away from the patient, or toward the user. Spatially relative terms such as “under”, “below”, “lower”, “over”, “upper”, and the like, are used for ease of description to explain the positioning of one element relative to a second element. These terms are intended to encompass different orientations of the device in addition to different orientations than those depicted in the figures. Further, terms such as “first”, “second”, and the like, are also used to describe various elements, regions, sections, etc and are also not intended to be limiting. Like terms refer to like elements throughout the description.

As used herein, the terms “having”, “containing”, “including”, “comprising” and the like are open ended terms that indicate the presence of stated elements or features, but do not preclude additional elements or features. The articles “a”, “an” and “the” are intended to include the plural as well as the singular, unless the context clearly indicates otherwise.

The present invention may be carried out in other specific ways than those herein set forth without departing from the scope and essential characteristics of the invention. The present embodiments are, therefore, to be considered in all respects as illustrative and not restrictive, and all changes coming within the meaning and equivalency range of the appended claims are intended to be embraced therein.

What is claimed is:

1. A cannula for insertion into a patient comprising:
a distal section with an outer cylindrical surface;
a second section adjacent the distal section and including an outer cylindrical surface, the second section including a smaller outer diameter than the distal section;
a shoulder formed at a junction of the distal section and the second section; and
a coating applied to the outer cylindrical surface of the second section, an outer diameter of the coating being less than or equal to the outer diameter of the distal section.

2. The cannula of claim 1, wherein the distal section includes a tip on a distal end.

3. The cannula of claim 1, wherein the distal section includes a tapered section that increases in size from a smaller first longitudinal position to a proximally-located larger second longitudinal position.

4. The cannula of claim 1, wherein the shoulder is aligned substantially perpendicular to a longitudinal centerline that extends through the distal and second sections.

5. The cannula of claim 1, wherein the coating contacts the shoulder.

6. The cannula of claim 1, wherein the second section includes a greater length than the distal section.

7. The cannula of claim 1, further comprising a hollow interior that extends through the distal and second sections.

8. A cannula for insertion into a patient comprising:
a tapered first section that extends between a tip with a reduced outer diameter to a shoulder with an enlarged outer diameter;
a second section adjacent and extending proximally outward from the first section with a second section outer diameter that is less than the enlarged outer diameter of the shoulder;
a coating applied to an outer surface of the second section and in contact with the shoulder, a coating outer diameter being less than or equal to the enlarged outer diameter of the shoulder; and
a hollow interior that extends through the first and second sections.

9. The cannula of claim 8, wherein the shoulder is formed at a junction of the first and second sections.

10. The cannula of claim 9, wherein the shoulder is substantially perpendicular with a longitudinal centerline of the first and second sections.

11. The cannula of claim 8, wherein the coating outer diameter is substantially constant along a length of the second section.

12. The cannula of claim 8, wherein the first section is continuously tapered from the tip to the shoulder.

13. A cannula for insertion into a patient comprising:
a tapered distal section with an outer cylindrical surface;
a second section adjacent the distal section and including an outer cylindrical surface, the intermediate section including a smaller outer diameter than the distal section; and
a coating applied to the outer cylindrical surface of the intermediate section, an outer diameter of the coating being less than or equal to the outer diameter of the distal section.

14. The cannula of claim 13, further including a shoulder formed at a junction of the distal section and the second section and wherein the coating contacts the shoulder.

15. The cannula of claim 14, wherein the shoulder is substantially straight and aligned substantially perpendicular to a longitudinal centerline that extends through the distal and second sections.

16. The cannula of claim 13, further comprising a non-tapered section positioned distally of the second section.

17. The cannula of claim 13, wherein the distal section includes a tip.

18. A cannula for insertion into a patient comprising:
a tapered first section;
a second section extending proximally from the first section;
a shoulder formed at a junction between the first and second sections, the shoulder including a larger outer diameter than the second section;
a coating applied to an outer surface of the second section and in contact with the shoulder, a coating outer diameter being less than or equal to the outer diameter of the shoulder; and
a hollow interior that extends through the first and second sections.

19. The cannula of claim 18, wherein the first section is tapered from a tip to the shoulder.

20. The cannula of claim 18, wherein the shoulder is aligned substantially perpendicular to a longitudinal centerline that extends through the distal and second sections.

21. A cannula for insertion into a patient comprising:
a cylindrical tapered tip;
a second cylindrical section adjacent the tip, the second cylindrical section including a smaller outer diameter than a proximal end of the tip; and
a coating applied to an outer surface of the second cylindrical section, an outer diameter of the coating being less than or equal to the outer diameter of the proximal end of the tip.

22. The cannula of claim 21, further comprising a shoulder formed at a junction of the cylindrical tapered tip and the second cylindrical section, the shoulder aligned in a non-parallel manner with a longitudinal centerline that extends through the cylindrical tapered tip and the second cylindrical section.

23. The cannula of claim 21, wherein the cylindrical tapered tip includes a continuous taper.

24. A cannula for insertion into a patient comprising:
a hollow cylindrical body including a first tapered section and an adjacent non-tapered second section, the first tapered section positioned at a distal end of the body;
a shoulder formed at a proximal end of the first tapered section, the shoulder extending between an outer surface of the first tapered section and an outer surface of the second section; and
a coating applied to the outer surface of the second section, an outer diameter of the coating being less than or equal to an outer diameter of the proximal end of the first tapered section.

25. The cannula of claim 24, wherein the coating is spaced away from the shoulder.

26. The cannula of claim 24, wherein the first tapered section includes a continuous taper.
